(19) United States
(12) Patent Application Publication
(10) Pub. No.: US 2010/0143571 A1
(43) Pub. Date: Jun. 10, 2010

(54) PROCESS FOR PRODUCTION OF OMEGA-3 RICH MARINE PHOSPHOLIPIDS FROM KRILL

(76) Inventor: Harald Breivik, Porsgrunn (NO)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/515,098

(22) PCT Filed: Nov. 15, 2007
(86) PCT No.: PCT/NO07/00402

§ 371 Date: Feb. 17, 2010

Related U.S. Application Data
(60) Provisional application No. 60/859,289, filed on Nov. 16, 2006.

(51) Int. Cl. A23L 1/325; A23K 1/10; A23K 1/18; C11B 1/10; C07C 45/78; C08B 37/08

(52) U.S. Cl. 426/643; 426/417; 554/21; 568/366; 536/20

(57) ABSTRACT

The present disclosure relates to a process for preparing a substantially total lipid fraction from fresh krill, a process for separating phospholipids from the other lipids, and a process for producing krill meal.

### FIELD OF THE INVENTION

The present invention relates to a process for preparing a substantially total lipid fraction from fresh hill, and a process for separating phospholipids from the other lipids. The invention also relates to a process for production of high quality krill meal.

### BACKGROUND OF THE INVENTION

Marine phospholipids are useful in medical products, health food and human nutrition, as well as in fish feed and means for increasing the rate of survival of fish larval and fry of marine species like cod, halibut and turbot.

Phospholipids from marine organisms comprise omega-3 fatty acids. Omega-3 fatty acids bound to marine phospholipids are assumed to have particularly useful properties.

Products such as fish milt and roe are traditional raw materials for marine phospholipids. However, these raw materials are available in limited volumes and the price of said raw materials is high.

Krill are small, shrimp-like animals, containing relatively high concentrations of phospholipids. In the group Euphasiids, there is more than 80 species, of which the Antarctic krill is one of these. The current greatest potential for commercial utilisation is the Antarctic Euphausia superba. E. superba has a length of 2-6 cm. Another Antarctic krill species is E. crystallorphias. Meganyctiphanes norvegica, Thysanoessa inermis and T. raschii are examples of northern krill.

Fresh hill contains up to around 10% of lipids, of that approximately 50 of % phospholipids in Euphausia superba. Phospholipids from krill comprise a very high level of omega-3 fatty acids, whereof the content of eicosapentaenoic acid (EPA) and docosahexaenoic acid (DHA) is above 40%. The approximate composition of lipids from the two main species of Antarctic krill is given in Table 1.

TABLE 1
Composition of krill lipids. Lipid classes,
(approximate sum EPA + DHA)

| | | | | Ratio |
|---|---|---|---|---|
| | Wax esters | Glycerides | Phospholipids | EPA/DHA |
| Euphausia | 1 | 50 (7) | 50 (40-45) | 1.4-1.5 |
| superba | | | | |
| Euphausia | 40 | 20 (4) | 40 (30-33) | 1.3 |
| crystallorphias | | | | |

Furthermore, Antarctic krill has lower level of environmental pollutants than traditional fish oils.

The krill has a digestive system with enzymes, including lipases that are very active around 0° C. The lipases stay active after the krill is dead, hydrolysing part of the krill lipids. An unwanted effect of this is that krill oil normally contains several percents of free fatty acids. If the krill has to be cut into smaller fragments before being processed, the person skilled in the art will immediately realise that this will increase the degree of hydrolysis. Thus, it is a desire to find a process that can utilise whole, fresh krill, or whole body parts from krill, as such a process will provide a product with improved quality and low degree of hydrolysis of lipids. This improved quality will affect all groups of krill lipids, including phospholipids, triglycerides and astaxanthin esters.

Krill lipids are to a large extent located in the animals' head. A process that can utilise fresh krill is therefore also well suited for immediate processing of the by-products from krill wherefrom the head is peeled off, a product that can be produced onboard the fishing vessel.

From U.S. Pat. No. 6,800,299 of Beaudion et al. it is disclosed a method for extracting total lipid fractions from krill by successive extraction at low temperatures using organic solvents like acetone and ethanol. This process involves extraction with large amounts of organic solvents which is unfavourable.

K. Yamaguchi et al. (J. Agric. Food Chem. 1986 34, 904-907) showed that supercritical fluid extraction with carbon dioxide, which is the most common solvent for supercritical fluid extraction, of freeze dried Antarctic krill resulted in a product mainly consisting of unpolar lipids (mostly triglycerides), and no phospholipids. Yamaguchi et al. reported that oil in krill meal was deteriorated by oxidation or polymerisation to such an extent that only limited extraction occurred with supercritical CO₂. Y. Tanaka and T. Ohkubo (J. Oleo. Sci. (2003), 52, 295-301) quotes the work of Yamaguci et al. in relation to their own work on extraction of lipids from salmon roe. In a more recent publication (Y. Tanaka et al. (2004), J. Oleo. Sci., 53, 417-424) the same authors try to solve this problem by using a mixture of ethanol and CO₂ for extracting the phospholipids. By using CO₂ with 5% ethanol no phospholipids were removed from freeze dried salmon roe, while by adding 10% ethanol, 30% of the phospholipids were removed, and by adding as much as 30% ethanol, more than 80% of the phospholipids were removed. Freeze drying is a costly and energy consuming process, and not suited for treatment of the very large volumes of raw materials that will become available by commercial krill fisheries.

Tanaka et al. tried to optimise the process by varying the temperature of the extraction, and found that low temperatures gave the best results. 33° C., a temperature just above the critical temperature for CO₂, was chosen as giving best results.

Contrary to these findings, we have surprisingly found a process for extraction of a substantially total lipid fraction from fresh krill, without the need for complicated and costly pre-treatment like freeze drying of large volumes. The lipid fraction contained triglycerides, astaxanthin and phospholipids. We did not have to dry or deoil the raw material before processing. Contrary to Tanaka et al. we have found that a short heating of the marine raw material was positive for the extraction yield. It was also shown that pre-treatment like a short-time heating to moderate temperatures, or contact with a solid drying agent like molecular sieve, of the krill can make ethanol wash alone efficient in removing phospholipids from fresh krill.

### SUMMARY OF THE INVENTION

It is a main object of the present invention to provide a process for preparing a substantially total lipid fraction from fresh krill without using organic solvents like acetone.

The exposure to the fluid under supercritical pressure will prevent oxidation from taking place, and the combined carbon dioxide/ethanol is expected to deactivate any enzymatic hydrolysis of the krill lipids. As the process according to the invention requires a minimum of handling of the raw materials, and is well suited to be used on fresh hill, for example onboard the fishing vessel, the product according to the invention is expected to contain substantially less hydrolysed and/or oxidised lipids than lipid produced by conventional processes. This also means that there is expected to be less deterioration of the krill lipid antioxidants than from conventional processing. The optional pre-treatment involving short-time heating of the fresh krill will also give an inactivation of enzymatic decomposition of the lipids, thus ensuring a product with very low levels of free fatty acids.

Another object of the present invention is to provide a process for preparing a substantially total lipid fraction from other marine raw materials like fish gonads, Calanus species, or high quality krill meal.

Another object of the present invention is to provide a substantially total lipid fraction high in long chain polyunsaturated omega-3 fatty acids.

These and other objects are obtained by the process and lipid fraction as defined in the accompanying claims.

According to the invention it is provided a process for extracting a substantially total lipid fraction from fresh krill, comprising the steps of:

a) reducing the water content of krill raw material; and
b) isolating the lipid fraction.

Optionally, the above-mentioned process comprising a further step of:

a-1) extracting the water reduced krill material from step a) with CO₂ at supercritical pressure containing ethanol, methanol, propanol or iso-propanol. This step, a-1), is performed directly after step a).

In a preferred embodiment of the invention it is provided a process for extracting a substantially total lipid fraction from fresh krill, comprising the steps of:

a) reducing the water content of krill raw material;
a-1) extracting the water reduced krill material from step a) with CO₂ containing ethanol, the extraction taking place at supercritical pressure; and
b) isolating the lipid fraction from the ethanol.

In a preferred embodiment of the invention, step a) comprises washing of the krill raw material with ethanol, methanol, propanol and/or iso-propanol in a weight ratio 1:0.5 to 1:5. Preferably, the krill raw material is heated to 60-100° C., more preferred to 70-100° C., and most preferred to 80-95° C., before washing. Furthermore, the krill raw material is preferably heated for about 1 to 40 minutes, more preferred about 1 to 15 minutes, and most preferred for about 1 to 5 minutes, before washing.

In another preferred embodiment of the invention, step a) comprises bringing the krill raw material in contact with molecular sieve or another form of membrane, such as a water absorbing membrane, for removal of water.

Preferably, the amount of ethanol, methanol, propanol and/or iso-propanol in step a-1) is 5-20% by weight, more preferably 10-15% by weight.

In addition to producing a product containing the total lipids of krill, the invention also can be used for separating phospholipids from the other lipids. To separate the total lipids obtained by extraction at supercritical pressure, according to the present invention into the different lipid classes, extraction of the said total lipids with pure carbon dioxide can remove the non-polar lipids from the omega-3 rich phospholipids. Extraction of the total lipids with carbon dioxide containing less than 5% ethanol or methanol is another option.

As the phospholipids are much richer in the valuable omega-3 fatty acids than the other lipid classes, this makes the invention useful for producing high concentrates of omega-3 fatty acids. While commercially available fish oils contain 11-33% total omega-3 fatty acids (Hjaltason, B and Haraldsson, G G (2006) Fish oils and lipids from marine sources, In: Modifying Lipids for Use in Food (FD Gunstone, ed), Woodhead Publishing Ltd, Cambridge, pp. 56-79), the phospholipids of krill contain much higher levels (Ellingsen, T E (1982) Biokjemiske studier over antarktisk krill, PhD thesis, Norges tekniske høyskole, Trondheim. English summary in Publication no. 52 of the Norwegian Antarctic Research Expeditions (1976/77 and 1978/79)), see also Table 1. The omega-3 rich phospholipids can be used as they are, giving the various positive biological effects that are attributed to omega-3 containing phospholipids. Alternatively, the phospholipids can be transesterified or hydrolysed in order to give esters (typically ethyl esters) or free fatty acids or other derivatives that are suitable for further concentration of the omega-3 fatty acids. As examples, the ethyl esters of krill phospholipids will be valuable as an intermediate product for producing concentrates that comply with the European Pharmacopoeia monographs no. 1250 (Omega-3-acid ethyl ester 90), 2062 (Omega-3-acid ethyl esters 60) and 1352 (Omega-3-acid triglycerides). At the same time, the remaining lipids (astaxanthin, antioxidants, triglycerides, wax esters) can be used as they are for various applications, including feed in aquaculture, or the lipid classes can be further separated.

Thus, still another object of the present invention is to provide a process for separating phospholipids from the other lipids as described above.

Another object of the invention is to produce a high quality krill meal. As the lipids are removed at an initial step of the process, the meal will be substantially free of oxidised and polymerised lipids. This will make the meal very well suited for applications where it is important to avoid oxidative stress, i.e. for use in aquaculture feed, especially starting feed for marine fish species. The krill meal of the present invention is thus well suited for feeding fish larvae and fry, as well as fish and crustaceans. Furthermore, the krill meal of the invention may be used as a source for production of high quality chitosan.

### DETAILED DESCRIPTION OF THE INVENTION

The process can be performed with a wide variety of processing conditions, some of which are exemplified below.

In the following “fresh” krill is defined as krill that is treated immediately after harvesting, or sufficiently short time after harvesting to avoid quality deterioration like hydrolysis or oxidation of lipids, or krill that is frozen immediately after harvesting. Fresh krill can be the whole krill, or by-products from fresh krill (i.e. after peeling). Fresh krill can also be hill, or by-products from krill, that have been frozen shortly after harvesting.

Moreover “krill” also includes krill meal.

### BRIEF DESCRIPTION OF THE FIGURES

FIG. 1 shows a picture of E. superba used as raw material for extraction.

FIG. 2 shows the material after extraction as described in Example 7 below.

### EXAMPLES

### Example 1

### Processing of Freeze Dried Krill

Freeze dried krill was extracted with CO₂ at supercritical pressure. This gave a product of 90 g/kg. Analysis showed that the extract contained a sum of EPA plus DHA of only 5.4%, showing that this did not contain a significant amount of the omega-3 rich phospholipids. A second extraction with CO₂ containing 10% ethanol resulted in an extract of 100 g/kg (calculated from starting sample weight). ³¹P NMR showed that the product contained phospholipids. The extract contained a sum of EPA plus DHA of 33.5%.

In both steps the extraction conditions were 300 bar, 50° C.

Thus, it is possible substantially to separate the omega-3 rich phospholipids from the less omega-3 rich components of the krill lipids.

In a second experiment the freeze dried krill was extracted twice with the same pressure and temperature as above, first with 167 parts (weight) of pure CO₂, and then with 167 part (weight) of CO₂ containing 10% ethanol. The combined extract (280 g/kg raw material) was analysed by ¹³C and ³¹P NMR. The analyses showed that the product contained triglycerides and phospholipids as major components. Like the previous extracts the dark red colour showed that the extract contained astaxanthin.

We are not aware that a process according to Example 1 has been used for freeze dried hill. It could be argued that this could be anticipated from Y. Tanaka et al. (2004) J. Oleo Sci. 53, 417-424. However, in this prior art CO₂ with 10% ethanol resulted in only 30% of the phospholipids being extracted. 20% ethanol had to be used in order to extract 80% of the phospholipids.

### Examples According to the Invention

### Example 2

Fresh E. superba (200 g) was washed with ethanol (1:1, 200 g) at around 0° C. The ethanol extract (1.5%) contained inorganic salts (mainly NaCl) and some organic material.

The ethanol washed krill was extracted with CO₂ containing 10% ethanol. This gave an extract of 12 g (6% based on starting krill). Analysis (TLC and NMR) showed that the extract contained phospholipids, triglycerides and astaxanthin.

The person skilled in the art will realise that carbon dioxide at supercritical pressure can act as a solvent for ethanol. Thus, an alternative procedure for modifying the solvent power of the CO₂ is to utilise pressure/temperature conditions so that ethanol is dissolve directly from the ethanol containing krill raw material, without having to be added by a pre-treatment of the CO₂. This also applies for the examples below.

### Example 3

Fresh E. superba (200 g) was washed with ethanol (1:3, 600 g) at around 0° C. The ethanol extract (7.2%) contained phospholipids, triglycerides and astaxanthin, and some inorganic salts. The extract contained 26.3% (EPA+DHA), showing that the relative content of phospholipids was high.

The ethanol washed krill was extracted with CO₂ containing 10% ethanol. This gave an extract of 2.2% based on starting krill. Analysis (TLC and NMR) showed that the extract contained phospholipids, triglycerides and astaxanthin. However, as the extract contained only 8.1% (EPA+DHA) it was concluded that the phospholipids content was low.

### Example 4

Fresh E. superba was treated with the same two-step process as above, except that the ethanol amount in the washing step was increased to 4:1. The ethanol extract was 7.2% compared to the starting material, while the supercritical fluid extract was 2.6%.

### Example 5

Fresh E. superba (200 g) was put in contact with molecular sieve (A3, 280 g) in order to remove water from the krill raw material. Extraction with CO₂ containing 10% ethanol gave an extract of 5.2% calculated from the starting weight of krill. Analyses showed that the extract contained triglycerides, phospholipids and astaxanthin. The extracted whole krill was completely white, except for the black eyes.

Example 5 shows the effect of removing water. Molecular sieve was chosen as an alternative to ethanol. These examples are not intended to be limiting with regard to potential agents for removal of water. Molecular sieve and other drying agents can be mild and cost effective alternatives to freeze drying.

### Example 6

Fresh E. superba (200 g) was washed with ethanol (1:1) as in example 2, but with the difference that the raw material had been pre-treated at 80° C. for 5 minutes. This gave an ethanol extract of 7.3%. Supercritical fluid extraction with CO₂ containing 10% ethanol gave an additional extract of 2.6% calculated from the fresh raw material. The total extract was 9.9%, and analyses (TLC, NMR) showed that the extract was rich in phospholipids, and also contained triglycerides and astaxanthin. The remaining, whole krill was completely white, except for the black eyes.

### Example 7

Fresh E. superba (12 kg) was heated to 80° C. for a few minutes and thereafter extracted with ethanol (26 kg). This gave an ethanol extract of 0.82 kg (7%). Analysis of lipid classes (HPLC; Column: Alltima HP silica 3 μm; detector: DEDL Sedere; Solvents: Chloroform/methanol) showed a content of 58% phospholipids. Analysis by GC (area %) showed a content of 24.0% EPA and 11.4% DHA, sum EPA+DHA=35.4%.

The remaining krill was extracted at 280 bar and 50° C. with CO₂ (156 kg) containing ethanol (15 kg). This gave an extract of 0.24 kg (2%). The remaining krill was white, except for the dark eyes. Analysis of lipid classes showed a content of 19% phospholipids. The extract contained 8.9% EPA and 4.8% DHA (sum 13.7%). Extraction of the remaining krill material (Folch method) showed a content of only 0.08 kg lipids (0.7% compared to initial krill weight). This means that substantially all lipids had been extracted.

### Example 8

Fresh E. superba (12 kg) was extracted with ethanol (33 kg) without heat treatment. This gave an extract of 0.29 kg (2.4%). Analysis of lipid classes as above showed a content of 28.5% phospholipids.

The results show that heat-treatment gives an increased yield of lipids compared to the same treatment with no heating. After heat-treatment of the raw material, one part (weight) of ethanol gave the same result as four parts of ethanol without heat treatment. Also, heating gave an ethanol extract that was more rich in phospholipids and omega-3 fatty acids than when the ethanol treatment was performed without heating.

The heating times in the examples should not be limiting for the invention. The person known in the art will realise that exact heating times are difficult to monitor for large volumes of biological material. Thus, the heating time may vary depending of the amount of krill that is to be processed at a specific time. Also, the temperature used for pre-heating is not limited to the temperature given in the examples. Experiments showed that pre-heating to 95° C. tended to increase the yield of lipids in step a) even higher than pre-heating to 80° C. Also, for large volumes of krill it may be difficult to obtain exactly the same temperature in all the krill material.

The heat treatment gives as additional result that the highly active krill digestive enzymes are inactivated, reducing the potential lipid hydrolysis.

### Example 9

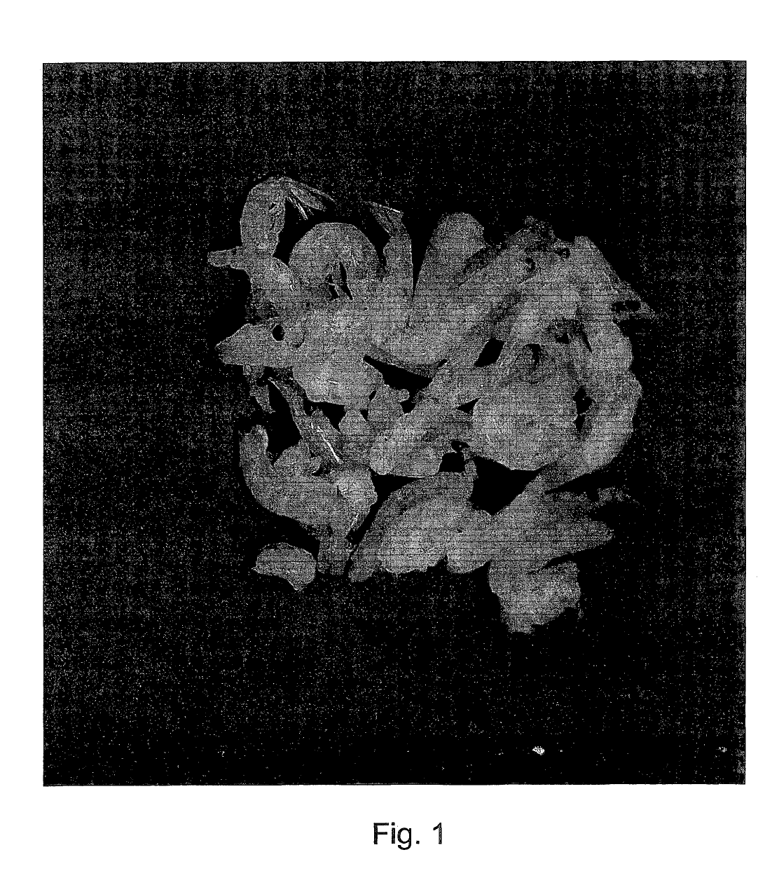

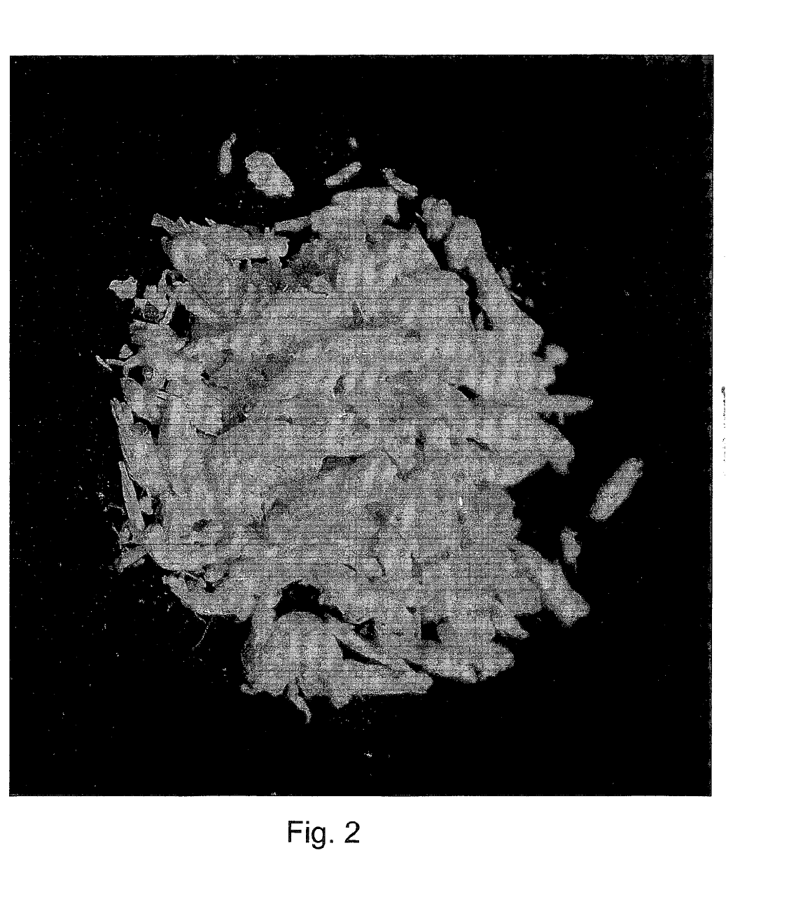

FIG. 1 shows a picture of E. superba used as raw material for extraction. FIG. 2 shows the material after extraction as described in Example 7. The other examples gave very similar material after extraction. The extracted krill is dry, and can easily be made into a powder, even manually by pressing between the fingers. The de-fatted powder contains proteins as well as chitosan and other non-lipid components from the krill. The powders smell similar to dry cod. As this powder is substantially free of lipids, it will give a meal substantially without oxidised polyunsaturated fatty acids. This is very different from krill meal produced according to traditional processes, where substantially all of the phospholipid fraction will be remain in the meal, giving rise to oxidised and polymerised material. Krill meal produced according to the present process will thus give much reduced oxidative stress compared to traditional krill meal or fish meal when used in feed for aquaculture. The krill meal will also be very suitable in feed for crustaceans, including lobster, and for feeding wild-caught King Crabs (Paralithodes camtschatica) in order to increase the quality and volume of the crab meat. As the meal is substantially free of polymerised lipids, it will also be beneficial for production of high quality chitosan, and for other processed where a high quality meal is needed.

Because the krill lipids oxidises very rapidly, and become less soluble in common solvents, the person skilled in the art will realise that a similar high quality krill meal could not be obtained by de-fatting of traditional krill meal, for example by use of organic solvents.

The person skilled in the art will realise that the processes described above also can be used for other raw materials than krill, for example the isolation of omega-3 rich phospholipids from fish gonads, or from Calanus species. Some krill species are rich in wax esters (example: E. crystallorphias), and the same will be the case for Calanus species. The person skilled in the art will realise that by processing as described above, the wax esters will be concentrated in the unpolar lipid fractions.

Furthermore, the person skilled in the art will realise that combination of process steps as given above can be used for separating the polar (i.e. phospholipids) and unpolar lipids of krill. It will also be possible to make an extract of the total lipids of krill according to one of the examples above, and then make a second extraction of this intermediary product in order to separate the lipid classes. For example, an extraction with pure carbon dioxide would remove the non-polar lipids from the omega-3 rich phospholipids.

In another embodiment, the process according to the invention is used to extract krill meal, wherein provided the krill meal has been produced in a sufficiently mild way to avoid deterioration of the krill lipids.

The person skilled in the art will also realise that a process as described above can be used to extract other marine raw materials like fish gonads and Calanus species.

A lipid fraction, or lipid product, derived from the process according to the invention may have some additional advantages related to quality compared to known hill oil products (produced by conventional processes), such as for instance a krill oil from Neptune Biotechnologies & Bioresources extracted from a Japanese krill source (species not specified) with the following composition:

| Total Phospholipids | ≧40.0% | |
|---|---|---|
| Esterified astaxanthin | ≧1.0 | mg/g |
| Vitamin A | ≧1.0 | IU/g |
| Vitamin E | ≧0.005 | IU/g |
| Vitamin D | ≧0.1 | IU/g |

| | |
|---|---|
| Total Omega-3 | ≧30.0% |
| EPA | ≧15.0% |
| DHA | ≧9.0% |

A lipid product or fraction according to the invention is expected to;
contain substantially less hydrolysed and/or oxidised lipids than lipid produced by conventional processes,
be less deterioration of the krill lipid antioxidants than from conventional processing,
contain very low levels of free fatty acids, and/or
be substantially free from trace of organic solvents.

By “oxidised” lipids is meant both primary oxidation products (typically measured as peroxide value), secondary oxidation products (typically carbonyl products, often analysed as anisidine value) and tertiary oxidation products (oligomers and polymers).

Thus, the invention includes commercial lipid or krill oil products produced by one of the processes according to the invention.

Products like, for instance, the dietary supplement, Superba™ (Aker BioMarine, Norway), might be produced by a process according to the present invention.

The person skilled in the art will realise that the quality of a product produced by a process of the present invention will be improved compared to a product produced by traditional extraction of krill meal.

Moreover, examples of a lipid compositions obtained by the process according to the invention are presented in the tables below, and also included herein.

TABLE 2

| Lipid composition | |
|---|---|
| Phospholipids | >30-40% by weight |
| EPA | >5-15% by weight |
| DHA | >5-15% by weight |

According to the invention, the extract can be concentrated with respect to the content of phospholipids. Some typical lipid compositions are illustrated by table 3-5, and included herein:

TABLE 3

| Lipid composition | |
|---|---|
| Phospholipids | ≧50% by weight |
| EPA | ≧15% |
| DHA | ≧10% |

As can be seen from Example 7, a lipid composition as described in Table 3 can also be obtained by only applying extraction according to step a) of the invention.

TABLE 4

| Lipid composition | |
|---|---|
| Phospholipids | ≧80% by weight |
| EPA | ≧20% |
| DHA | ≧13% |

TABLE 5

| Lipid composition | |
|---|---|
| Phospholipids | ≧90% by weight |
| EPA | ≧23% |
| DHA | ≧15% |

The invention shall not be limited to the shown embodiments and examples.

1. A process for extracting a substantially total lipid fraction from fresh krill, comprising the steps of:
a) reducing the water content of krill raw material by washing with at least one alcohol chosen from ethanol, methanol, propanol, and iso-propanol in a weight ratio of krill raw material:at least one alcohol ranging from 1:0.5 to 1:5; and
b) isolating the lipid fraction from the at least one alcohol.

2. (canceled)

3. The process of claim 1, wherein at least one alcohol is ethanol.

4. The process of claim 1, further comprising a step:
a-1) extracting the water-reduced krill material from step a) with CO₂ at supercritical pressure comprising at least one alcohol chosen from ethanol, methanol, propanol and iso-propanol;
wherein step a-1) occurs immediately after step a).

5. The process of claim 1, wherein the krill raw material is heated at a temperature ranging from 60-100° C. before washing.

6. The process of claim 5, wherein the krill raw material is heated at a temperature ranging from 70-100° C. before washing.

7. The process of claim 6, wherein the krill raw material is heated at a temperature ranging from 80-95° C. before washing.

8. The process of claim 5, wherein the krill raw material is heated for about 1 to 40 minutes before washing.

9. The process of claim 8, wherein the krill raw material is heated for about 1 to 15 minutes before washing.

10. The process of claim 8, wherein the krill raw material is heated for about 1 to 5 minutes before washing.

11. (canceled)

12. (canceled)

13. The process of claim 4, wherein the amount of the at least one alcohol in step a-1) is 5-20% by weight.

14. The process of claim 13, wherein the amount of alcohol in step a-1) is 10-15% by weight.

15. A substantially total lipid fraction according to claim 1, wherein the lipid fraction comprises at least one of triglycerides, astaxanthin, and phospholipids, and is substantially free from oxidized lipids.

16. (canceled)

17. A medicament or food supplement comprising the substantially total lipid fraction according to claim 15.

18. A process for separating phospholipids from other lipids, comprising extracting the total lipid fraction obtained by the process of claim 1 with pure carbon dioxide, or carbon dioxide comprising less than 5% alcohol chosen from ethanol, methanol, propanol and iso-propanol.

19. The phospholipids fraction obtainable by the process of claim 18.

20. The phospholipids fraction of claim 19, wherein the phospholipids are further transesterified or hydrolysed.

21. The phospholipids fraction of claim 19, wherein the concentration of omega-3 fatty acids is at least 40% by weight.

22. A process for producing krill meal, comprising extracting a substantially total lipid fraction according to the process of; claim 1, and isolating the remaining krill raw material.

23. A krill meal substantially free of oxidised polyunsaturated fatty acids and other lipids according to claim 22.

24. An animal feed comprising the meal of claim 23.

25. An aquaculture feed comprising the krill meal of claim 23.

26. The aquaculture feed of claim 25, suitable for feeding at least one marine fish species.

27. The aquaculture feed of claim 25, suitable for feeding crustaceans.

28. High quality chitosan comprising the krill meal of claim 23.

29. The aquaculture feed of claim 26, suitable for feeding at least one of fish larvae and fish fry.
